(19) United States
(12) Patent Application Publication
(10) Pub. No.: US 2006/0064036 A1
(43) Pub. Date: Mar. 23, 2006

(54) Variable flexibility wire guide

(75) Inventors: Thomas A. Osborne, Bloomington, IN (US); Aaron Barr, Ellettsville, IN (US)

Correspondence Address: BRINKS HOFER GILSON & LIONE, P.O. BOX 10395, CHICAGO, IL, 60610, US

(73) Assignee: Cook Incorporated

(21) Appl. No.: 10/946,416

(22) Filed: Sep. 21, 2004

(51) Int. Cl. A61M 25/00

(52) U.S. Cl. 600/585

(57) ABSTRACT

The present invention provides a wire guide having a wire core and a braided sheath. The wire core includes a proximal end and distal end, wherein the braided sheath is attached to the distal end of the wire core and serves as a flexible pulling section. The braided sheath is woven of a plurality of strands and may be made of various material based on the application, such as stainless steel, a shape memory alloy, or a radiopaque material. The wire guide has a flexible tip at the proximal end opposite the flexible pulling section. A stiff section is provided between the flexible tip and the flexible pulling section to allow manipulation of the wire guide through a body lumen. Proximate the distal end of the wire core a tapered section is provided to increase flexibility of the wire guide toward the distal end. The braided sheath is received over and attached to the wire core. In addition, a shoulder is provided in the wire core providing a smooth transition from the wire core to the braided section. The braided sheath extends from the shoulder beyond the distal end of the wire core.

### BACKGROUND

1. Field of the Invention

The present invention generally relates to a medical surgical device and specifically a wire guide for percutaneous placement providing variable flexibility along its length.

2. Description of Related Art

Wire guides are widely used throughout the medical industry. Wire guides are used for advancing intraluminal devices such as stent delivery catheters, balloon dilation catheters, atherectomy catheters, and the like within body lumens. Typically, the wire guide is positioned inside the inner lumen of an introducer catheter. The wire guide is advanced out of the distal end of the introducer catheter into the patient until the distal end of the wire guide reaches the location where the interventional procedure is to be performed. After the wire guide is inserted, another device such as a stent and stent delivery catheter is advanced over the previously introduced wire guide into the patient until the stent delivery catheter is in the desired location. After the stent has been delivered, the stent delivery catheter can then be removed from a patient by retracting the stent delivery catheter back over the wire guide. The wire guide may be left in place after the procedure is completed to ensure easy access if it is required. Conventional wire guides include an elongated wire core with one or more tapered sections near the distal end to increase flexibility. Generally, a flexible body such as a helical coil or tubular body is disposed about the wire core. The wire core is secured to the flexible body at the distal end by soldering, brazing or welding which forms a rounded distal tip. In addition, a torquing means is provided on the proximal end of the core member to rotate, and thereby steer a wire guide having a curved tip, as it is being advanced through a patient's vascular system.

A major requirement for wire guides and other intraluminal guiding members, is that they have sufficient stiffness to be pushed through the patient's vascular system or other body lumen without kinking. However, they must also be flexible enough to pass through the tortuous passageways without damaging the blood vessel or any other body lumen through which they are advanced. Efforts have been made to improve both the strength and the flexibility of wire guides in order to make them more suitable for their intended uses, but these two properties tend to be diametrically opposed to one another in that an increase in one usually involves a decrease in the other.

For certain procedures, such as when delivering stents around challenging take-off, tortuosities, or severe angulation, substantially more support and/or vessel straightening is frequently needed from the wire guide. Wire guides have been commercially available for such procedures which provide improved support over conventional wire guides. However, such wire guides are not very steerable and in some instances are so stiff they can damage vessel linings when being advanced.

In other instances, extreme flexibility is required as well. For example, when branched or looped stents are to be delivered to a branched vascular region, it is beneficial to insert the wire guide from the branch where a stent is to be located. However, the stent may need to be introduced and guided from a separate branch. In this situation, the wire guide is inserted into the patient's vascular system near the desired stent location and a grasping device is inserted in the branch from which the stent will be introduced. The wire guide may be advanced back along the branch to provide the grasping device access to the distal end of the wire guide. However, the wire guide should be extremely flexible to allow grasping and manipulation of the wire guide without damaging the tissue around the bifurcation formed by the luminal branch. Further, the wire guide should be extremely kink resistant to avoid damaging the wire guide as it is grasped. After the wire guide is retrieved by the grasping device, the stent may be delivered over the wire guide to the desired location. However, available wire guides are not designed to provide the flexibility required to cross up and over the bifurcation of the luminal branch and yet also provide the stiffness required to aid in the insertion of the stent.

In view of the above, it is apparent that there exists a need for an improved design for a wire guide.

### SUMMARY OF THE INVENTION

In satisfying the above need, as well as, overcoming the enumerated drawbacks and other limitations of the related art, the present invention provides a wire guide having a wire core and a braided sheath. The braided sheath is attached to a first end of the wire core and serves as a flexible pulling section. The braided sheath is woven of a plurality of strands and may be made of various material based on the application, such as stainless steel, a shape memory alloy, or a radiopaque material. The wire guide also has a flexible tip opposite the flexible pulling section. A stiff section is provided between the flexible tip and the flexible pulling section to allow manipulation of the wire guide through a body lumen.

Toward the first end of the wire core, a tapered section is provided to increase flexibility of the wire guide over the flexible pulling section. The braided sheath is received over the wire core and is attached to the wire core by solder or adhesive. In addition, a shoulder is provided in the wire core facilitating a smooth transition from the wire core to the braided sheath. The braided sheath extends from the shoulder beyond the end of the wire core, thereby forming the flexible pulling section.

The flexible tip is provided opposite the flexible pulling section, near the second end of the wire core and includes a tapered section reducing the diameter of the wire core toward the flexible tip. A coil member is disposed about the second end and attached to the wire core. A sleeve, such as, a polyurethane layer surrounds the wire core and the braided sheath to improve kink resistance and guidablity of the wire guide. In addition, a lubricous coating is provided over the sleeve to improve the ease of advancement of the wire guide through the patient's vascular system. The lubricous coating may be a hydrophilic coating and may be omitted from the flexible pulling section to improve graspability of the braided sheath.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a partial sectional view of a wire guide embodying the principles of the present invention;

FIG. 2 is a cross sectional view of an aneurysm illustrating the insertion of a stent graft delivery system and a wire guide embodying the principles of the present invention;

FIG. 3 is a cross sectional view of an aneurysm illustrating the stent graft delivery system and the wire guide being advanced therefrom;

FIG. 4 is a cross sectional view of an aneurysm illustrating a snare pulling the wire guide across the bifurcation between the femoral branches;

FIG. 5 is a cross sectional view of an aneurysm illustrating the side branch stent graft being partially unsheathed;

FIG. 6 is a cross sectional view of an aneurysm illustrating the delivery sheath and dilator for the side branch extension stent graft being introduced over the wire guide;

FIG. 7 is a cross sectional view of an aneurysm illustrating the delivery sheath for the side branch extension stent graft being advanced through the side branch stent graft;

FIG. 8 is a cross sectional view of an aneurysm illustrating the wire guide being pulled out of the side branch stent graft delivery sheath to free the arm of the side branch stent graft;

FIG. 9 is a cross sectional view of an aneurysm illustrating the deployment of the side branch stent graft;

FIG. 10 is a cross sectional view of an aneurysm illustrating the deployment of the side branch extension stent graft;

FIG. 11 is a cross sectional view of an aneurysm illustrating the deployment of the main body stent graft over the wire guide;

FIG. 12 is a cross sectional view of an aneurysm illustrating a completed stent graft installation with all delivery systems removed;

FIG. 13 is a partial sectional view of a wire guide having a coil member along its length and embodying the principles of the present invention.

### DETAILED DESCRIPTION OF THE INVENTION

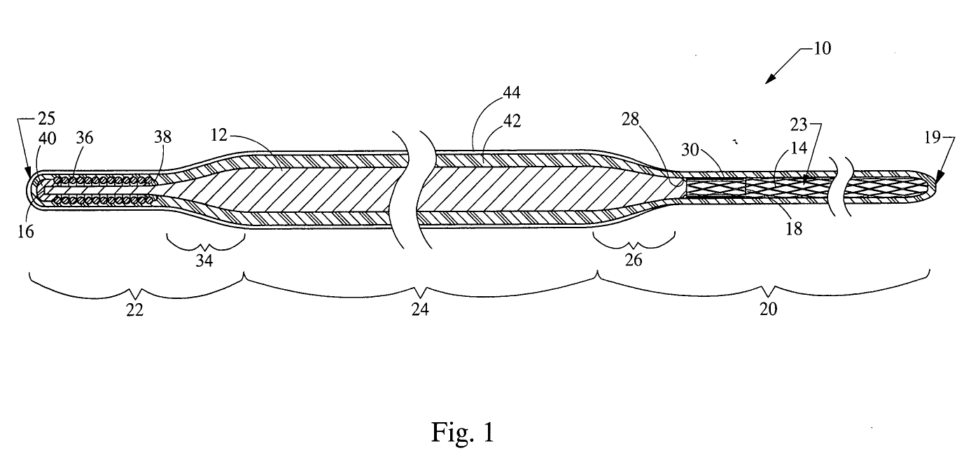

Referring now to FIG. 1, a wire guide embodying the principles of the present invention is illustrated therein and designated at 10. The wire guide 10 includes a core member, such as, a wire core 12 and a braided member, such as, braided sheath 14. The braided sheath 14 and a portion of the wire core 12 cooperate to form a flexible pulling section 20 near a first end 19 of the wire guide 10. Opposite the flexible pulling section 20 is a flexible tip section 22 located near a second end 25 of the wire guide 10. Between the flexible pulling section 20 and the flexible tip section 22, is a stiff middle section 24.

Each of the three sections 20, 22, and 24 are particularly beneficial for inserting a stent around a branched or looped body lumen. Sometimes, it is beneficial to insert the wire guide 10 from the branch where the stent is to be located, however, the stent may need to be introduced and guided from a separate branch. The first end 19 of the wire guide 10 is inserted into the patient's vascular system near the desired stent location. Similarly, a grasping device can be inserted in another branch from which the stent will be introduced. The wire guide 10 is advanced back along the branch to provide the grasping device access to the first end 19 of the wire guide 10. However, the wire guide 10 must be extremely flexible to allow grasping and manipulation of the first end 19 without damaging the tissue around the bifurcation formed by the luminal branch. Accordingly, the braided sheath 14 provides the needed flexibility in the flexible pulling section 20 of the wire guide 10. The flexible pulling section 20 may be retrieved by the grasping device through the entry in other branch. The flexible tip section 22 is pulled into the patient and the stiff middle section 24 is used to manipulate the flexible tip section 22 to a location of interest. The described configuration provides access for other devices to be advanced along the wire guide 10 to the location of interest.

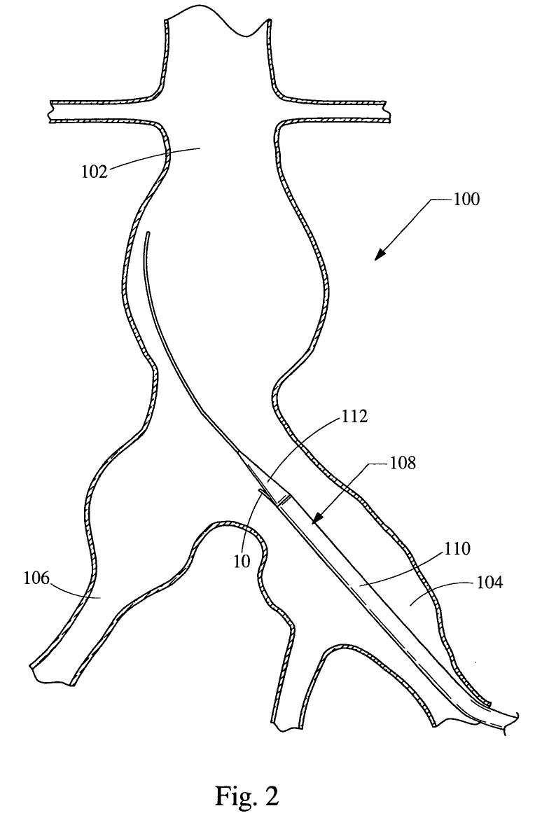

A detailed example of such a procedure is illustrated in FIGS. 2-12. An arterial aneurysm 100 extends from the aorta 102 into a first femoral branch 104 and a second femoral branch 106.

FIG. 2 shows the side branch stent graft and delivery system 108 inserted and positioned near the target side branch artery. The wire guide 10 of this invention is shown protruding slightly from between the delivery sheath 110 and the inner dilator 112. The dilator 112 has a small groove to accommodate the wire guide 10.

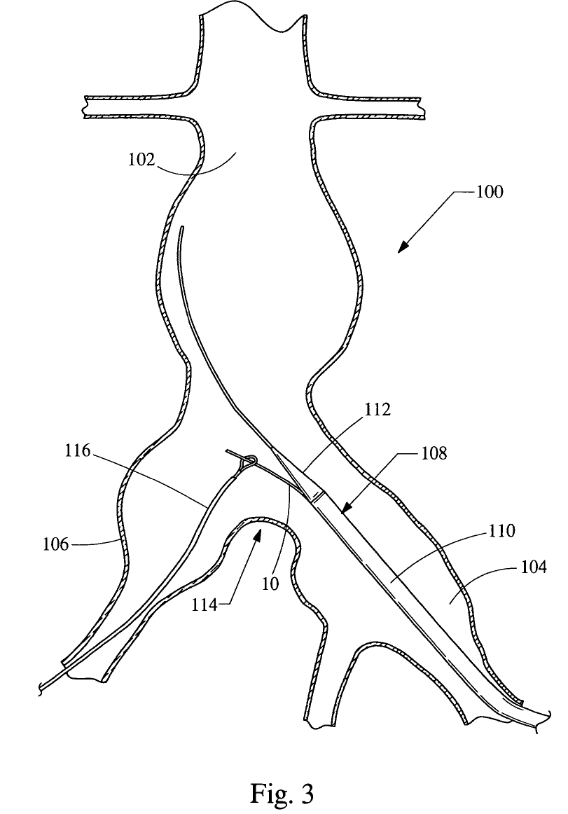

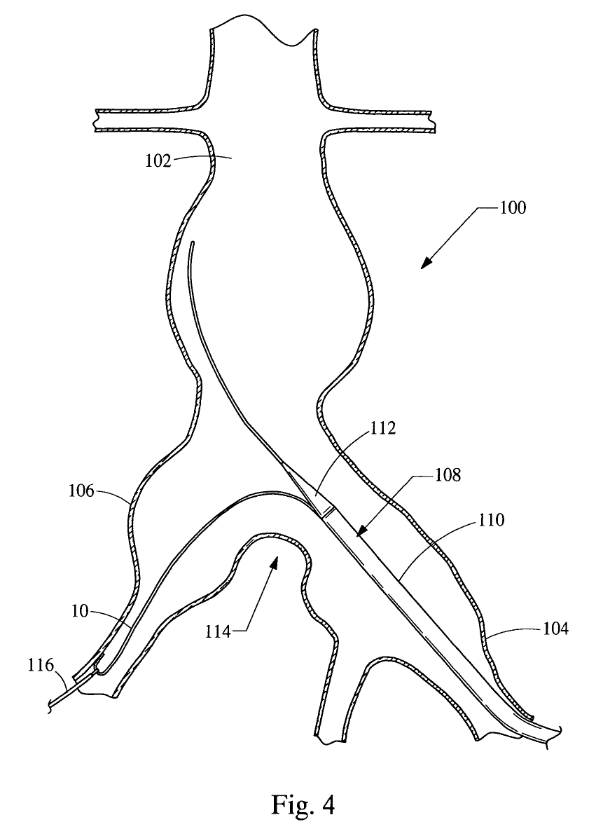

Now referring to FIGS. 3 and 4, the wire guide 10 must be snared, pulled to the opposite side entry site and pulled out of the entry site to a point external to the patient. FIG. 3 shows the wire guide 10 of this invention advanced a few centimeters to provide enough length of wire so that the snare 116 can securely capture and pull the wire guide 10 over the bifurcation 114 and out the snare entry site. In this maneuver, the end of the wire guide 10 is folded, or doubled over as it is pulled by the snare 116 through the artery and out the entry site of the snare 116. This requires that the wire guide 10 be very flexible in this section so as to not traumatize the artery wall while making a very small radius fold. Further, the wire guide 10 must be strong enough to withstand the tensile forces of the pulling through process and not be permanently kinked or deformed such that, the side branch extension delivery system can be loaded onto the wire guide 10 once the end has been pulled out.

Ordinary wire guide construction is not suitable for these requirements. The small, “safety” wires used in conventional flexible tip wire guides do not have suitable tensile strength to insure that the wire will not break allowing the coil to unravel or stretch, thereby becoming unusable. The use of the fine wire braid as a safety wire increases the tensile strength of the “safety” wire and does not add appreciable stiffness. Typical safety wires are small round or rectangular wires, 0.003 to 0.005 in. diameter or 0.002 by 0.004 in. rectangular with tensile strengths in the range of 2 to 10 pounds pull strength. The multiple fine wire braid material can have a tensile strength from 10 to 25 pounds pull strength.

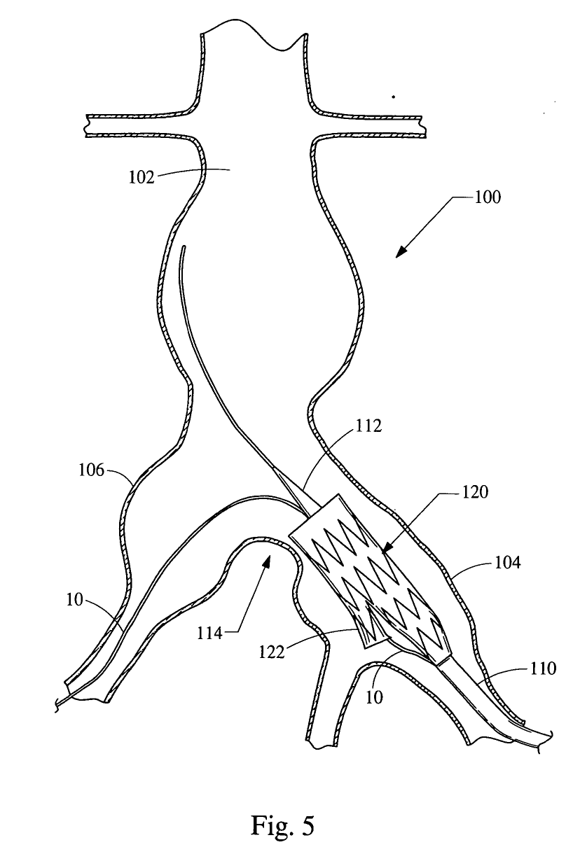

Now referring to FIGS. 4 and 5, the wire guide 10 used in this procedure must be pulled across the bifurcation between the femoral arteries 106, 104 and the aorta 102. FIG. 4 shows the wire guide 10 snared and pulled over the bifurcation 114 and toward the (entry site for the snare 116) on the opposite side. The artery wall around the bifurcation 114 is very thin and fragile due to the aneurismal disease that the stent grafts are attempting to repair. Therefore, the body of the wire guide 10 needs to be smooth and slippery. Typical wire guides are coils with stiffening central cores or mandrels. The surface of a coil type wire guide is “bumpy” due to the successive coils along the length of the wire guide. Pulling this type of surface across tissue can result in abrasion of the diseased or damaged tissue, increasing the risk of aneurism rupture during the repair procedure. The wire guide 10 of this invention uses a smooth body portion to protect the artery wall in the area of the bifurcation. The smooth, non-traumatic surface can be achieved by eliminating the outer coil portion and increasing the diameter of the coil portion an appropriate amount, then coating the body portion with a soft polymer material such as polyurethane, then coating the polymer with a lubricious, hydrophilic coating to lower the coefficient of friction between the artery wall the body of the wire guide 10. In addition, the wire guide 10 must be stiff enough to provide guidance or direction for the side branch extension stent graft delivery system. Normal percutaneous entry wire guides are not stiff enough to control and deflect a device as bulky and stiff as a stent graft delivery system.

FIG. 5 shows the wire guide 10 of this invention pulled across the bifurcation 114 and out the snare entry site on the opposite side. The side branch stent graft 120 has been partially unsheathed, exposing the short side branch leg of the stent graft 120. The wire guide 10 of this invention still passes through the side branch stent graft 120 through the short arm 122 and back into the sheath 110.

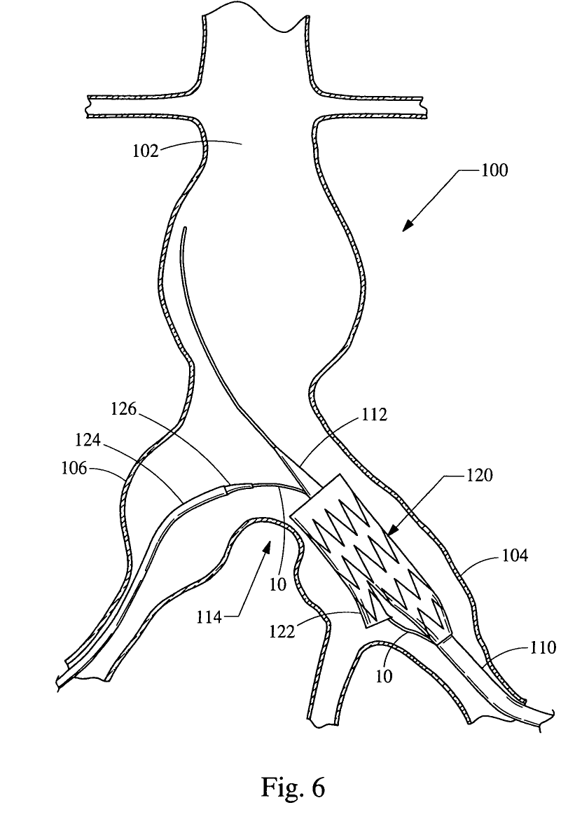

FIG. 6 shows the delivery sheath 124 and dilator 126 for the side branch extension stent graft being introduced and advanced over the wire guide 10 of this invention from the opposite side.

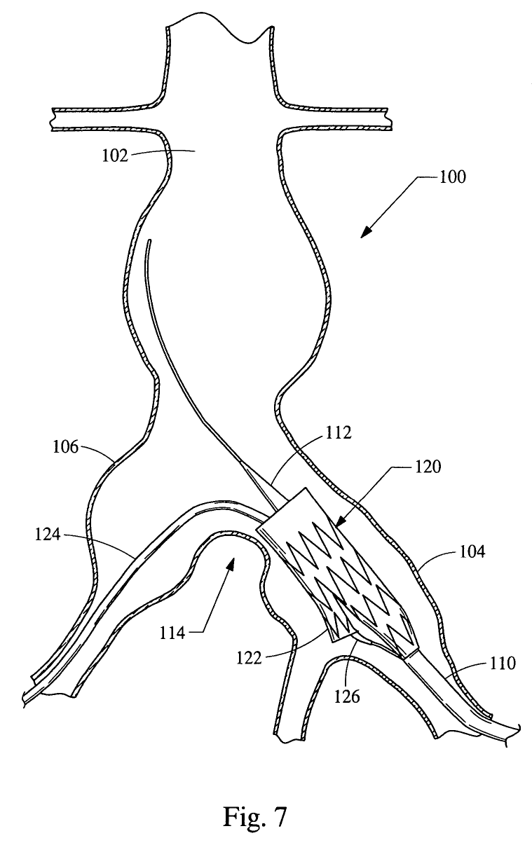

FIG. 7 shows the delivery sheath 124 and dilator 126 for the side branch extension stent graft being advanced through the side branch stent graft 120 all the way to the point where the wire guide 10 of this invention enters the delivery sheath 110 of the side branch stent graft 120.

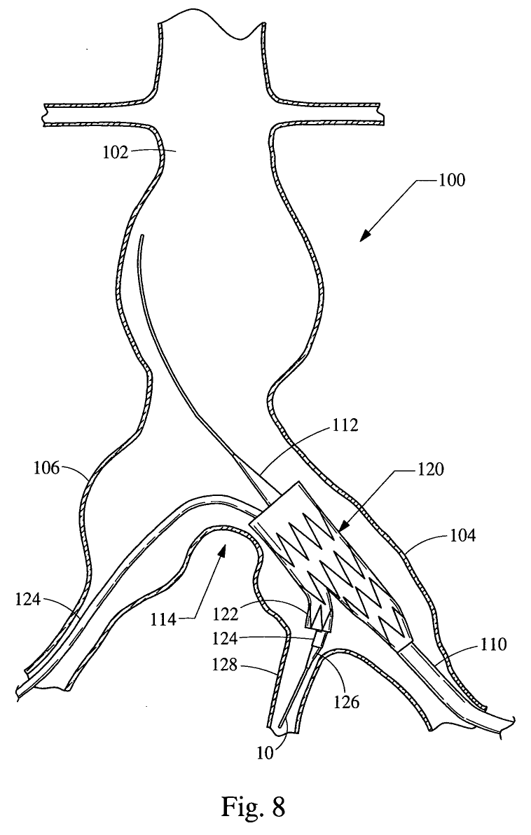

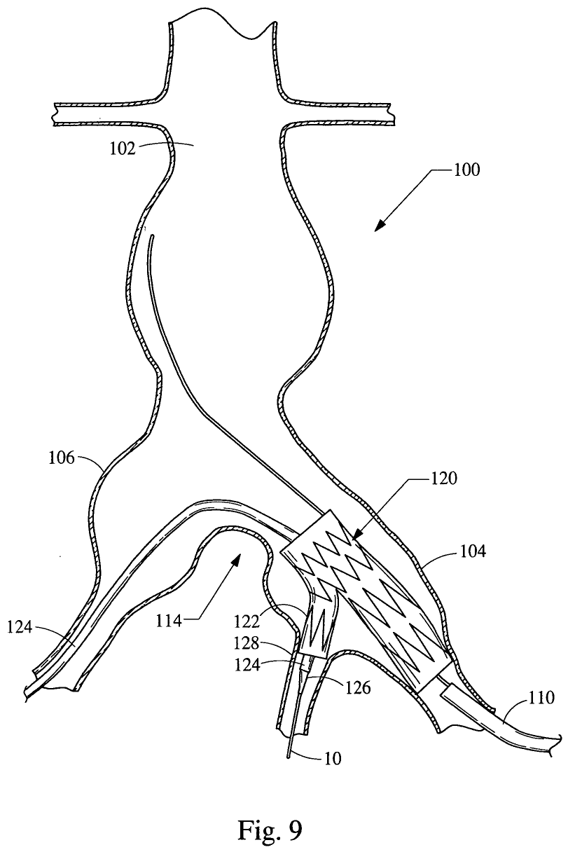

Now referring to FIGS. 8 and 9, the wire guide 10 of this invention must also have a flexible portion at the opposite end located in the target branch 104. This is the end of the wire that is used to enter the target side branch artery 128 where the extension stent graft 130 is to be placed. If the end of the wire guide 120 that is being advanced into the side branch artery is stiff, the physician will not be able to direct the wire into the desired artery and the end of the wire would be traumatic and damage artery wall as it is advanced along the artery.

FIG. 8 shows the wire guide 10 of this invention pulled out through the side branch extension stent graft delivery system 108 until the opposite end of the wire guide 10 exits the distal end of the side branch stent graft delivery sheath 110, freeing the short arm 122 of the stent graft 120 and allowing the wire guide 10 of this invention to be advanced with the delivery sheath 124 through the short arm extension stent graft into the target side branch artery 128.

FIG. 9 shows the side branch stent graft delivery sheath 110 withdrawn, completing the deployment of the side branch stent graft 120.

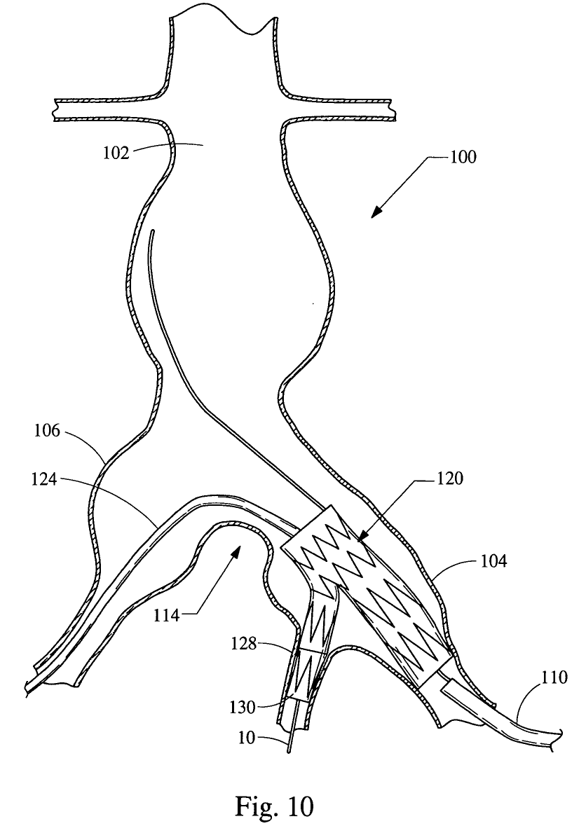

FIG. 10 shows the short arm extension stent graft 130 delivered and deployed over the wire guide 10 of this invention. The wire guide 10 of this invention and the extension stent graft delivery sheath 124 are still in place.

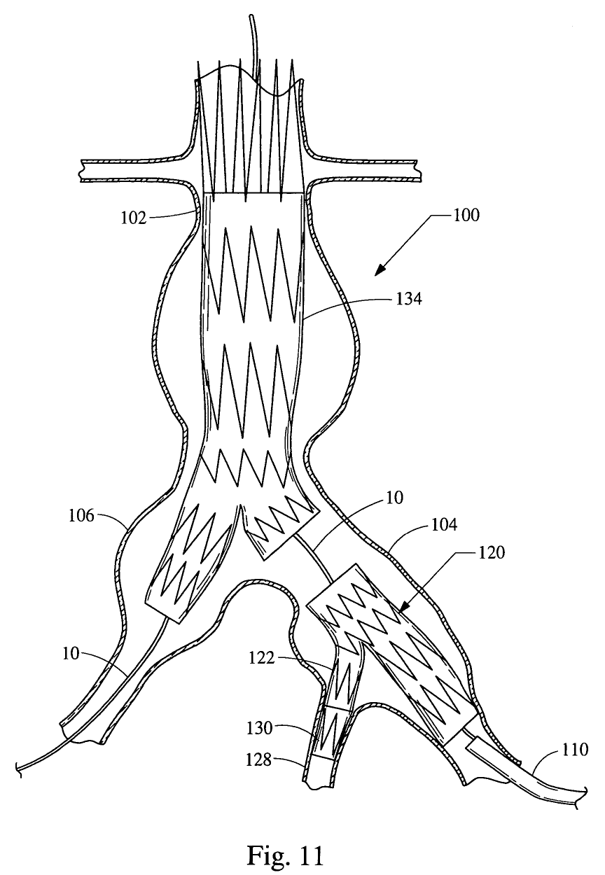

FIG. 11 shows the short arm extension stent graft delivery sheath 124 withdrawn and removed. The wire guide 10 of this invention has been withdrawn from across the bifurcation 114 and used for the delivery and deployment of the main body stent graft 134.

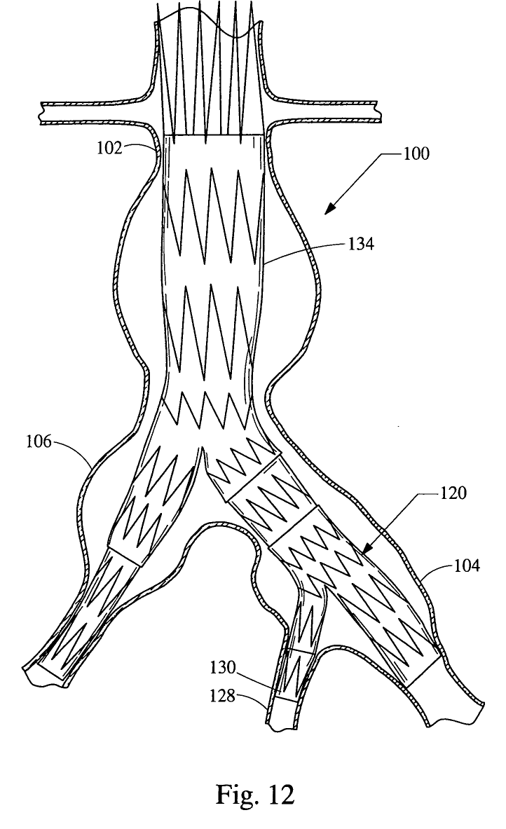

FIG. 12 shows the completed stent graft installation with all stent grafts in place and delivery systems removed.

Referring again to FIG. 1, additional flexibility is provided in the flexible pulling section 20 by a tapered section 24 that reduces the diameter of the wire core 12 towards a first end 18 of the wire core 12. In addition, the braided sheath 14 is attached to and extends from the first end 18 of the wire core 12. Preferably, the braided sheath 14 is received over and around the first end 18 and is attached to the wire core 12 by a bond 30 of solder or adhesive. A shoulder 28 is provided allowing the braided sheath 14 to seat against the shoulder 28. The radial height of the shoulder 28 is about the thickness of the braided sheath 14 thereby providing a smooth transition from the wire core 12 to the braided sheath 14 surrounding the first end 18. Further, the braided sheath 14 extends from the shoulder 28 beyond the first end 18 of the wire core 12.

The braided sheath 14 provides increased flexibility and kink resistance in combination with strength and graspability to provide benefits over other more common methods of providing wire guide flexibility. The braided sheath 14 is constructed of a plurality of strands 23 interwoven to provide strength to the braided sheath 14. The strands 23 are wrapped in a clockwise and counterclockwise direction, with strands weaving in and out of other strands. The density, thickness, or material of the strands may be varied to increase or decrease the flexibility along the braided sheath. The strands 23 are comprised of stainless steel or other common materials. Alternatively, the strands 23 may be comprised of Nitinol to provide increased control over the flexibility of the braid or a radiopaque material to provide increased visibility during grasping of the flexible pulling section 20.

The stiff middle section 24 allows the physician to direct the second end 25 of the wire guide 10 into sub-branches or further down the body lumen into which it was inserted. To provide improved control over flexibility of the wire guide 10, the wire core 12 is comprised of a shaped memory alloy, such as Nitinol. Alternatively, the wire core 12 may be constructed of commonly used wire guide material such as stainless steel.

To provide protection for the surrounding tissue as the second end 25 is being directed, the flexible tip section 22 is provided. The flexible tip section 22 includes a second tapered section 34. The second tapered section 34 reduces the diameter of the wire core 12 toward the second end 25 of the wire guide 10 thereby providing increased flexibility. A coil member 36 is disposed about the wire core 12. The coil member 36 is attached to the wire core 12 near the second tapered section 34 by solder joint 38 and at a second end 16 of the wire core 12 by a solder joint 40 that is formed into a rounded tip. The coil member 36 acts to control the flexibility of the wire core 12 along the flexible tip section 22. The coil 36 member is made of a radiopaque material, such as, platinum. Using a radiopaque material, allows for better visibility during manipulation of the wire guide 10.

The proportions of the flexible pulling section 20, stiff middle section 24, and flexible tip section 22 are also notable aspects of the wire guide 10. The wire guide 10 must be long and stiff enough to aid in the insertion of a stent, while being flexible enough and providing a long enough flexible pulling section 20 to allow the wire guide 10 to cross up and over the bifurcation of the branch, aiding in retrieval of the wire guide 10. Accordingly, for the delivery of a stent for treating aortic abdominal aneurism, the stiff middle section 24 is between about 50 and 200 cm in length, preferably about 100 cm, and having a core diameter of about 0.035 mm. The flexible pulling section 20 includes the first tapered section 26 and extends along the length of the braided sheath 14. The flexible pulling section 20 is between about 40 and 80 cm, preferably about 60 cm in length. Further, the first tapered section 26 is between about 5-15 cm in length, preferably between 8-10 cm; the distance from the first tapered section 26 to the distal end 18 of the wire core 12 is between about 5-15 cm, preferably about 10 cm; and the braided sheath 14 extends beyond the first end 18 of the wire core 12 by between about 30-50 cm, preferably about 40 cm. In addition the flexible tip 22 from the second tapered section 34 to the second end 16 of the wire core 12 is between about 3 and 5 cm in length. Although, these dimensions provide advantages for the above mentioned application, differing lengths are contemplated and may be more suitable for other applications. Further, certain aspects of the drawings such as the tapers may be exaggerated for illustrative purposes.

A sleeve 42 is disposed about the wire core 12 and the braided sheath 14 to provide to provide a smooth contiguous surface, so as not to damage the diseased tissue as the wire guide 10 is pulled over the bifurcation of the luminal branch. The sleeve 42 may be made of polyurethane or other commonly used sleeve materials to improve the performance of wire guides. In addition, a lubricous coating 44 is applied over the sleeve section 42. The lubricous coating 44 may be a hydrophilic coating to reduce surface friction, thereby improving the ease with which the wire guide 10 may be advanced through the body lumen. The hydrophilic coating may encompass the entire length of the wire guide 10, or alternatively, may encompass the wire core 12 but not the flexible pulling section 20 to provide improved graspability of the braided sheath 14.

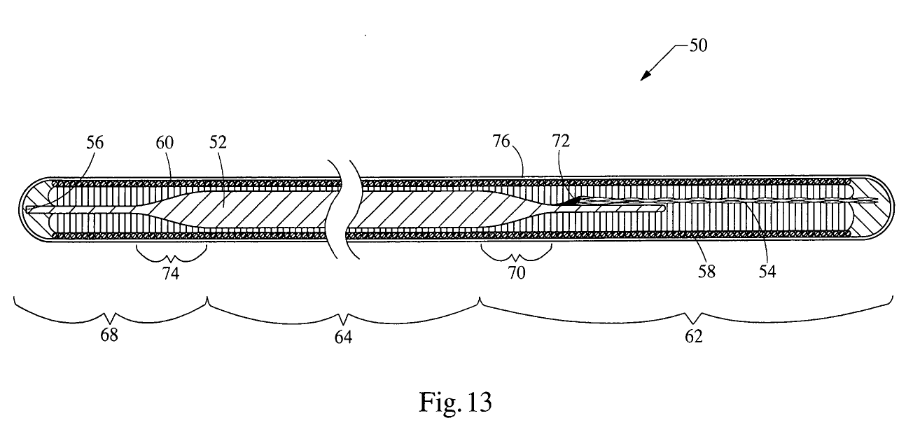

Now referring to FIG. 13, another embodiment of a wire guide 50 is provided having a wire core 52, braided sheath 54, and a coil member 60. The coil member 60 is attached to and disposed about the wire core 52 and braided sheath 54. Similar to the previous embodiment, the wire guide 50 has a flexible pulling section 62, a stiff middle section 64, and a flexible tip section 68.

The flexible pulling section 62 is formed by the wire core 52, the braided sheath 54, and the coil member 60. To provide the flexible pulling section 62, a tapered section 70 reduces the diameter of the wire core 52 towards a first end 58 providing additional flexibility. The braided sheath 54 is attached to the wire core 52 near the first end 58. Preferably, the braided sheath 54 is attached to the wire core 52 by a bond 72 of solder or adhesive. The braided sheath 54 is attached to the coil member 60 creating a mechanical link between the wire core 52 and the coil member 60. The braided sheath 54 may be attached to the coil member 60 by soldering, or other common attachment methods. The mechanical link between the wire core 52 and the coil member 60 provides tension to the coil member 60, while the flexibility of the braided sheath 54 results in increased flexibility along the flexible pulling section 62.

The stiff middle section 64 allows the physician to guide a flexible tip section 68 into sub-branches or further down the body lumen into which the wire guide 50 was inserted. To provide improved control over flexibility of the wire guide 50, the wire core 52 is comprised of a shaped memory alloy, such as, Nitinol. Alternatively, the wire core 52 may be constructed of commonly used wire guide material such as stainless steel.

To provide protection to vascular tissue as the flexible tip section 68 is being directed, the flexible tip section 68 includes a second tapered section 74. The second tapered section 74 reduces the diameter of the wire core 52 toward the proximal end 56 thereby providing increased flexibility. The wire core 52 is attached to the coil member 60 at a second end 56 of the wire core 52. The second end 56 may be attached to the coil member 60 by soldering or other common attachment methods.

Further, a friction reducing layer 76 is disposed about the coil member 60. The friction reducing layer 76 may be a sleeve or coating, such as, a Teflon coating to increase the ease, with which, the wire guide 50 may be advance through the patient's vascular system. In addition, the friction reducing layer 76 serves to provide a smooth outer diameter of the wire guide 50, so as not to damage the diseased tissue as the wire guide 50 is pulled over the bifurcation of the luminal branch.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

We claim:

1. A wire guide for introducing medical devices into a patient, the wire guide comprising:
a core member having a first and second end; and
a braided member woven of a plurality of strands, the braided member being affixed to the core member and extending from a first end of the core member to provide a flexible end section of the wire guide.

2. The wire guide of claim 1, wherein the braided member is soldered to the first end of the core member.

3. The wire guide of claim 1, further comprising an adhesive configured to attach the braided member to the first end of the core member.

4. The wire guide of claim 1, wherein a portion of the braided member is received over the core member.

5. The wire guide of claim 1, wherein the core member includes a shoulder and a height of the shoulder is about the thickness of the braided member thereby providing a smooth transition from the core member to the braided member.

6. The wire guide of claim 5, wherein the braided member is located around the core member and extends from the shoulder beyond the first end of the core member.

7. The wire guide of claim 1, further comprising a lubricious coating surrounding the core member.

8. The wire guide of claim 7, wherein the lubricious coating is a hydrophilic coating.

9. The wire guide of claim 1, wherein the core member includes a first tapered section proximal the braided member, the first tapered section being configured to increase flexibility toward the first end of the core member.

10. The wire guide of claim 9, wherein the core member includes a second tapered section, configured to increase flexibility toward the second end of the core member.

11. The wire guide of claim 10, wherein the core member includes a stiff section between the first tapered section and the second tapered section.

12. The wire guide of claim 11, wherein the stiff section is between about 50 to 200 cm in length.

13. The wire guide of claim 10, wherein a distance from the second tapered section to the second end of the core member is between about 3 and 5 cm.

14. The wire guide of claim 9, wherein a distance including the first tapered section and extending to an end of the braided member is between about 40 and 80 cm.

15. The wire guide of claim 9, wherein the first tapered section is between about 5 and 15 cm.

16. The wire guide of claim 9, wherein a distance from the first tapered section to the first end of the core member is between about 5 and 15 cm.

17. The wire guide of claim 1, wherein the braided member extends beyond the first end of the core member by a distance of between about 30 and 50 cm.

18. The wire guide of claim 1, further comprising a coil member disposed about the second end of the core member.

19. The wire guide of claim 18, wherein the coil member is a platinum coil.

20. The wire guide of claim 18, wherein the coil member is soldered to the core member.

21. The wire guide of claim 1, wherein the core member is comprised of a shape memory alloy.

22. The wire guide of claim 21, wherein the core member is comprised of Nitinol.

23. The wire guide of claim 1, wherein the plurality of strands are comprised of stainless steel.

24. The wire guide of claim 1, wherein the plurality of strands are comprised of a shape memory alloy.

25. The wire guide of claim 24, wherein the plurality of strands are comprised of Nitinol.

26. The wire guide of claim 1, wherein the plurality of strands are comprised of a radiopaque material.

27. The wire guide of claim 1, further comprising a coil member extending along the length of the core member.

28. The wire guide of claim 27, wherein braided member is attached to the coil member.

29. A wire guide for introducing medical devices into a patient, the wire guide comprising:
a core member having a first and second end;
a braided member woven of a plurality of strands, the braided member being affixed to the core member and extending from a first end of the core member to provide a flexible end section of the wire guide; and
a sleeve surrounding the core member and the braided member.

30. The wire guide of claim 29, wherein the sleeve is a polyurethane layer surrounding the core member and braided member.

31. The wire guide of claim 29, further comprising a lubricious coating surrounding the core member.

32. The wire guide of claim 31, wherein the lubricious coating is a hydrophilic coating.
